(19) United States
(12) Patent Application Publication
(10) Pub. No.: US 2021/0038778 A1
(43) Pub. Date: Feb. 11, 2021

(54) VACUUM SUCTION ACNE REMOVER WITH CAMERA FUNCTION

(71) Applicant: POWER 7 TECHNOLOGY CORP. (Shenzhen), Shenzhen (CN)

(72) Inventors: Mike CHEN, Shenzhen (CN); Yan-Qiu ZHANG, Shenzhen (CN)

(21) Appl. No.: 16/531,386

(22) Filed: Aug. 5, 2019

(51) Int. Cl. A61M 1/00; A61B 90/80; H04N 7/18; H04N 5/225

(52) U.S. Cl. CPC A61M 1/0066; A61B 90/80; H04N 7/183; B08B 5/04; H04N 5/2254; A61M 2205/3584; A61M 2205/8206; H04N 5/2253

(57) ABSTRACT

A vacuum suction acne remover includes a main control module, a vacuum suction module including a suction head and a vacuum pump connected to the suction head and controllable by the main control module to generate a suction force at the suction head for sucking out acne from the skin, and a camera module having an imaging lens controllable by the main control module to capture the image of the area being sucked by the vacuum suction module. During the suction process of the vacuum suction module, the camera module can observe the image of the suction area, and the suction of the vacuum suction module can be adjusted according to the depth of the acne displayed on the screen to avoid blind acne removing action and incomplete cleaning and to prevent the skin from being damaged due to excessive suction, thereby improving the efficiency and cleanliness of acne removal.

### 1. FIELD OF THE INVENTION

The present invention relates to intelligent storage technology and more particularly, to a vacuum suction acne remover with camera function, which uses a camera module to capture the image of the skin area being sucked by the vacuum suction module. Thus, during the suction process to remove the acne, the camera module can be used to observe the image of the suction area, and the suction force can be adjusted according to the depth of the acne displayed on the screen to avoid blind acne removing action and incomplete cleaning and to prevent the skin from being damaged due to excessive suction.

### 2. DESCRIPTION OF THE RELATED ART

Generally, blackheads are removed by instrumental suction. The instrumental suction method mainly removes blackheads from the skin through an electric blackhead remover. An electric blackhead remover includes a remover body and a suction head mounted on the top of the remover body. The housing accommodates therein a vacuum pump, a flexible air tube, a battery, a control circuit board and a LED circuit board. The vacuum pump is connected to the suction head by the flexible air tube. The control circuit board is electrically connected to the vacuum pump, the battery and the LED circuit board respectively. The LED circuit board has mounted thereon a plurality of LED lamps. The remover body is provided with a plurality of lamp holes that accommodate the LED lamps respectively. When the electric blackhead remover is used, the suction head is abutted against the opening of the skin follicle, and the control circuit board is controlled to turn on the vacuum pump, causing the vacuum pump to generate a vacuum suction force at the suction head for sucking out the blackhead from the skin follicle, thereby achieving the effect of cleaning.

The above electric blackhead remover can clean the blackheads to a certain extent. However, it is still not satisfactory in function. When cleaning the skin pores, it is often difficult for the user to see the cleanliness of the skin pores, so it is possible to stop the cleaning process before the grease dirt is completely removed, resulting in incomplete cleaning. In addition, it is also possible to continue the cleaning process after the grease dirt has been completely removed, causing the skin pore cleaning device to be used for too long and causing damage to the skin.

Therefore, how to solve the above-mentioned drawbacks and inconvenience is the direction that the relevant industry players engaged in this industry are eager to study and improve.

### SUMMARY OF THE INVENTION

The present invention has been accomplished under the circumstances in view. It is therefore the main object of the present invention to provide a vacuum suction acne remover with camera function, which comprises a main control module, a vacuum suction module comprising a suction head and a vacuum pump connected to the suction head and controllable by the main control module to generate a suction force at the suction head for sucking out an acne from the skin, and a camera module having an imaging lens controllable by the main control module to capture the image of the area being sucked by the vacuum suction module. During the suction process of the vacuum suction module, the camera module can be used to observe the image of the suction area, and the suction of the vacuum suction module can be adjusted according to the depth of the acne displayed on the screen to avoid blind acne removing action and incomplete cleaning and to prevent the skin from being damaged due to excessive suction, thereby improving the efficiency and cleanliness of acne removal.

According to another aspect of the present invention, the camera module is disposed inside an accommodation space of the vacuum suction module, and the imaging lens of the camera module is aligned with a suction port at the vacuum suction module, so that the surface of the skin can be directly and accurately observed. In turn, the visual deviation caused by the shooting angle is reduced, so that the process of acne adsorption can be accurately understood. Since the camera module is set inside the accommodation space, the original space can be effectively utilized without significantly increasing the overall volume.

According to still another aspect of the present invention, the suction head of vacuum suction module comprises a body member and a cap capped on the body member. The body member is provided with the detachable crystal on the side of the joint piece. The crystal is transparent, so the imaging lens of the camera module is not affected, and the crystal can be used to block the hole of the joint piece, preventing dirt from adhering to the imaging lens. Since the crystal can be disassembled, when the crystal is dirty, it can be disassembled for cleaning. After cleaning, the crystal is put back. Thus, cleaning the crystal is convenient.

Other advantages and features of the present invention will be fully understood by reference to the following specification in conjunction with the accompanying drawings.

### BRIEF DESCRIPTION OF THE DRAWINGS

FIG. 1 is an elevational view of a vacuum suction acne remover with camera function in accordance with the present invention.

FIG. 2 is an exploded view of the camera module and suction head of the vacuum suction acne remover with camera function in accordance with the present invention.

FIG. 3 is a block diagram of the present invention.

FIG. 4 is a block diagram of an alternate form of the present invention.

### DETAILED DESCRIPTION OF THE PREFERRED EMBODIMENT

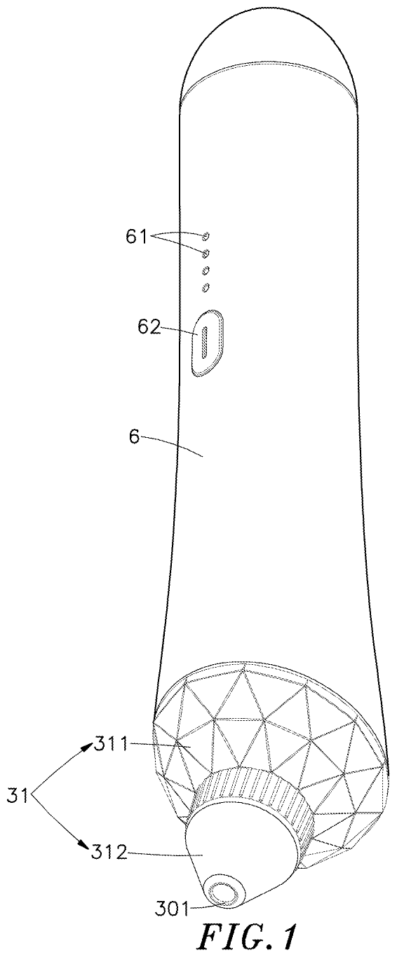

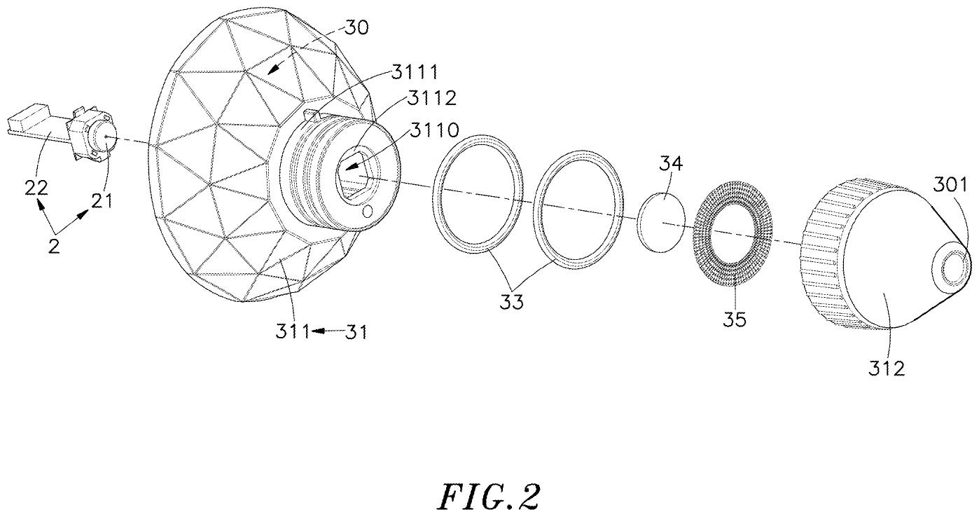

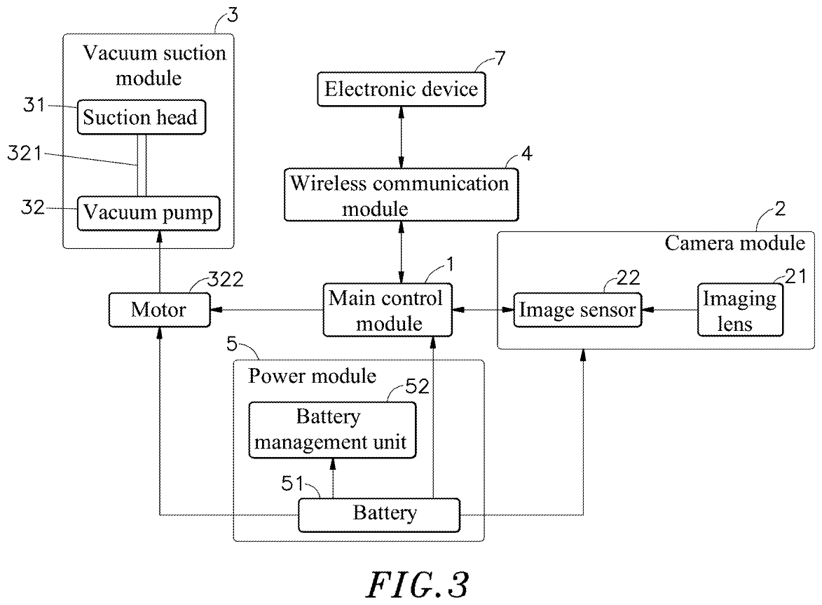

Referring to FIGS. 1-3, an elevational view of a vacuum suction acne remover with camera function in accordance with the present invention, an exploded view of the camera module and suction head of the vacuum suction acne remover with camera function in accordance with the present invention and a block diagram of the present invention are shown. As illustrated, the vacuum suction acne remover with camera function generally comprises a main control module 1, a camera module 2 and a vacuum suction module 3.

The main control module 1 is electrically connected with the camera module 2 and the vacuum suction module 3 and configured to control the camera module 2 and the vacuum suction module 3. The main control module 1 can be a multi-media image processing chip, the specific model is an AC1838DG3401 chip, and is connected with the camera module 2 by using an MIPI interface for collecting video data.

The camera module 2 comprises an imaging lens 21, and an image sensor 22 disposed at one side relative to the imaging lens 21 and electrically connected to the main control module 1.

The vacuum suction module 3 comprises a suction head 31, and a vacuum pump 32 connected to the suction head 31. The suction head 31 comprises a body member 311, and a cap 312 capped on the body member 311. The body member 311 and cap 312 of the suction head 31 define therein an accommodation space 30 for accommodating the camera module 2. Further, a suction port 301 is located on the cap 312 and disposed in alignment with the imaging lens 21 in the accommodation space 30 for capturing images. Further, a suction tube 321 is connected between the vacuum pump 32 and the cap 312 and can generate suction at the suction port 301.

The imaging lens 21 of the camera module 2 uses an optical imaging lens having a horizontal angle of view of 25 degrees and a focal length of 15 mm. The image sensor 22 of the camera module 2 uses a VGA level CMOS component and has multi-magnification function and is installed in the middle position of the suction head 31. It can observe the condition of the skin surface and the process of sucking out the acne from the skin, preventing blind acne removing action and incomplete cleaning.

Furthermore, the body member 311 of the suction head 31 is provided with a joint piece 3111 for positioning the cap 312. A hole 3110 is formed on the joint piece 3111 in communication with the accommodation space 30 for the placement of the imaging lens 21. Further, a groove 3112 is formed on the joint piece 3111 around the hole 3110. Further, rubber bands 33 are mounted on the outside of the joint piece 3111 of the body member 311 to provide a sealing and fixing function. Further, a crystal 34 is detachably mounted in the groove 3112 in joint piece 3111 to protect the imaging lens 21 against stains. Further, a dust screen 35 is mounted on the joint piece 3111 to block dust or impurities from entering the vacuum suction acne remover. Further, the shape of the cap 312 can be a boss shape, a cylinder shape or a square shape.

The vacuum pump 32 of the vacuum suction module 3 is mounted with a motor 322 that is electrically connected to the main control module 1. The main control module 1 can output PWM signals of different duty ratios to the motor 322, so that the motor 322 can adjust the suction force of the vacuum pump 32, thereby controlling the suction force of the suction head 31. In addition, the motor 322 is a 5V-powered motor with a maximum operating speed of 6500 RPM.

The above main control module 1 is further electrically connected with a wireless communication module 4, and the wireless communication module 4 can be used to wirelessly transmit the image of the suction area to the screen of an electronic device 7 such as a mobile phone, a tablet computer or a desktop computer, helping the user to observe the process of removing acne. The wireless communication module 4 can be at least one of a Bluetooth module, a WiFi module, or a 2.4G module. The Bluetooth module, the WiFi module, and the 2.4G module are common communication modes and can be produced according to actual requirements. If the wireless communication module 4 is a WiFi module, the WiFi protocol AP mode is adopted, and the mobile phone/tablet is directly connected to the device via WiFi. The support agreement includes but is not limited to 2.4G/5G IEEE802.11a/b/g/n/ac that can be purchased directly on the market.

In addition, the present invention further comprises a power module 5 electrically connected to the main control module 1, the camera module 2, and the vacuum pump 32. The power module 5 comprises a battery 51 that supplies power required by the main control module 1, the camera module 2 and the vacuum pump 32, and a battery management unit 52 electrically connected to the battery 51 to manage the operation of the battery 51. The battery 51 uses 18650 2000 mAh polymer battery of charging current 800 mA. It can charge the electronic device 7 through a transmission port 11. The model of the battery management unit 52 is IP5306, which is a model that can be purchased directly on the market.

The invention further comprises a housing 6. The housing 6 has at least one indicator light 61 and a button 62 mounted on the surface thereof and electrically connected to the main control module 1. The at least one indicator light 61 allows the user to instantly understand the suction level of the vacuum suction acne remover, and the button 62 allows the user to adjust the suction level.

When the present invention is actually used, the suction head 31 of the vacuum suction module 3 can be first placed against the skin, and the suction port 301 is aligned with the suction area where the acne needs to be removed. At this time, the vacuum pump 32 of the vacuum suction module 3 can generate a negative air pressure to suck out the acne from the skin in conjunction with the suction head 31. During the adsorption process OF suction head 31, the imaging lens 21 of the camera module 2 will take pictures of the acne suction area, and wirelessly transfer the picture to the screen (not shown) of the electronic device 7 (mobile phone, tablet computer or desktop computer) using the wireless communication module 4. The user can watch the screen and control the main control module 1 through the button 62 on the housing 6 according to the position of the acne displayed on the screen to adjust the power of the vacuum pump 32, thereby adjusting the suction of the suction head 31. In this way, it can avoid blind acne removing action and incomplete cleaning, and the user can observe the process of removing acne from the skin. So, the invention can accurately remove acne and prevent skin damage caused by excessive suction (such as redness and swelling), thereby improving the effect of removing acne.

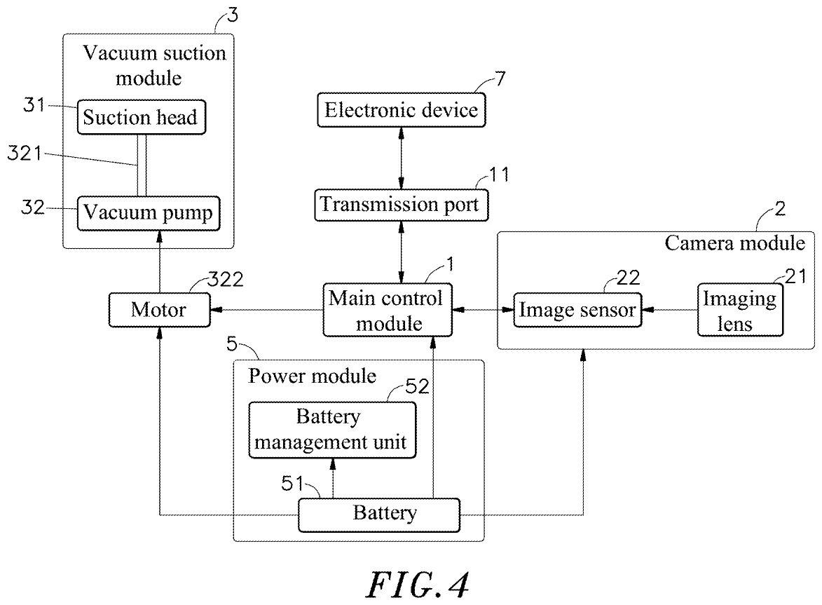

Referring to FIG. 4 that is a block diagram of an alternate form of the present invention, this alternate form eliminates the aforesaid wireless communication module 4, and the main control module 1 is provided with a structural design with a plurality of transmission ports 11, and the plurality of transmission ports 11 are respectively connected to the camera module 2 and the electronic device 7. The main control module 1 is a USB storage SOC chip, the specific model is Power7 US6808 chip. It adopts ARM926 architecture. The highest frequency is 192 MHz. It supports USB 2.0 high-speed communication and USB OHCI/EHCI host controller and transceiver. The transmission port 11 is a USB AF interface or a USB TYPE-C interface. It is compatible with different mobile phone systems. Further, it transmits data through a USB transmission line. The USB transmission line has one end thereof connected to the transmission port 11 and an opposite end thereof connected to the electronic device 7 (mobile phone, tablet computer or desktop computer) to realize data exchange and charging functions and to further overcome the problem of insufficient power generation due to long-term use.

The present invention has following advantages:

A. During the suction process of the vacuum suction module 3, the camera module 2 can be used to observe the image of the suction area, and the suction of the vacuum suction module 3 can be adjusted according to the depth of the acne displayed on the screen to avoid blind acne removing action and incomplete cleaning. Because it can observe the process of sucking out the acne from the skin, it can remove acne more accurately and reliably, and prevent the skin from being damaged due to excessive suction, thereby improving the efficiency and cleanliness of acne removal.

B. The camera module 2 is disposed inside the accommodation space 30 of the vacuum suction module 3, and the imaging lens 21 of the camera module 2 is aligned with the suction port 301 of the vacuum suction module 3, so that the surface of the skin can be directly and accurately observed. In turn, the visual deviation caused by the shooting angle is reduced, so that the process of acne adsorption can be accurately understood. Since the camera module 2 is set inside the accommodation space 30, the original space can be effectively utilized without significantly increasing the overall volume.

C. The body member 311 is provided with the detachable crystal 34 on the side of the joint piece 3111. The crystal 34 is transparent, so the imaging lens 21 of the camera module 2 is not affected, and the crystal 34 can be used to block the hole 3110 of the joint piece 3111, preventing dirt from adhering to the imaging lens 21. Since the crystal 34 can be disassembled, when the crystal 34 is dirty, it can be disassembled for cleaning. After cleaning, the crystal 34 is put back. Thus, cleaning the crystal 34 is convenient.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A vacuum suction acne remover, comprising a main control module, a camera module and a vacuum suction module, wherein:
said main control module is electrically connected with said camera module and said vacuum suction module;
said camera module comprises an imaging lens controllable by said main control module to capture an image of an area being sucked by said vacuum suction module;
said vacuum suction module comprises a suction head for sucking out an acne from the skin, and a vacuum pump connected to said suction head and controllable by said main control module to generate a suction force at said suction.

2. The vacuum suction acne remover as claimed in claim 1, wherein said main control module comprises at least one transmission port for transmitting the image captured by said camera module to an external electronic device.

3. The vacuum suction acne remover as claimed in claim 1, wherein said camera module further comprises an image sensor electrically disposed at one side relative to said imaging lens and electrically connected to said main control module and controllable by said main control module to capture the image of the area being sucked by said vacuum suction module via said imaging lens.

4. The vacuum suction acne remover as claimed in claim 1, wherein said suction head comprises a body member and a cap capped on said body member, said body member and said cap defining therein an accommodation space that accommodates said camera module, said cap comprising a suction port disposed in communication with said accommodation space and in alignment with imaging lens; said vacuum pump is provided with a suction tube that is connected to said suction port of said cap for enabling said vacuum pump to generate a suction force in said suction port.

5. The vacuum suction acne remover as claimed in claim 4, wherein said body member of said suction head comprises a joint piece for positioning said cap, said joint piece comprising a hole disposed in communication of said accommodation space for the placement of said imaging lens.

6. The vacuum suction acne remover as claimed in claim 4, wherein said joint piece further comprises a groove extended around said hole, and a crystal detachably mounted in said groove.

7. The vacuum suction acne remover as claimed in claim 4, further comprising at least one rubber band mounted on said joint piece of said body member.

8. The vacuum suction acne remover as claimed in claim 4, further comprising a dust screen mounted on said joint piece of said body member.

9. The vacuum suction acne remover as claimed in claim 1, wherein said vacuum suction module further comprises a motor mounted on said vacuum pump and electrically connected to said main control module.

10. The vacuum suction acne remover as claimed in claim 1, further comprising a wireless communication module electrically connected to said main control module for transmitting the image captured by said camera module to an external electronic device wirelessly.
